(19) United States
(12) Patent Application Publication
(10) Pub. No.: US 2018/0168511 A1
(43) Pub. Date: Jun. 21, 2018

(54) DISTALLY-FACING ELECTRODE ARRAY WITH LONGITUDINALLY MOUNTED SPLINES

(71) Applicant: Boston Scientific Scimed Inc., Maple Grove, MN (US)

(72) Inventors: Peter Hall, Andover, MN (US); Peter A. Ridl, Stillwater, MN (US)

(21) Appl. No.: 15/845,679

(22) Filed: Dec. 18, 2017

Related U.S. Application Data
(60) Provisional application No. 62/436,389, filed on Dec. 19, 2016.

(51) Int. Cl. A61B 5/00; A61B 5/042; A61B 18/14; A61M 25/00

(52) U.S. Cl. CPC A61B 5/6858; A61B 5/0422; A61B 2018/00267; A61M 25/0074; A61B 18/1492

(57) ABSTRACT

A catheter comprises an elongate catheter body extending longitudinally from a proximal end to a distal end, an expandable electrode assembly disposed at the distal end of the catheter body, the electrode assembly comprising a housing encasing a position sensor and a plurality of flexible splines extending from the distal end of the catheter body to a distal end of the housing, and a distal cap positioned over a distal end of the housing, the distal cap being configured to receive the distal end of the housing and to further receive distal ends of flexible splines of the plurality of flexible splines in longitudinal or about longitudinal directions relative to the catheter body. Distal portions of the flexible splines provide reduced stiffness as compared to more proximal portions. The expandable electrode assembly is configured to be transitioned between a collapsed configuration suitable for delivery and an expanded configuration.

### CROSS-REFERENCE TO RELATED APPLICATION

This application claims priority to Provisional Application No. 62/436,389, filed Dec. 19, 2016, which is herein incorporated by reference in its entirety.

### TECHNICAL FIELD

The present disclosure generally relates to electrode assemblies for use in cardiac procedures and more particularly, to an electrode assembly that may be utilized in a cardiac mapping procedure.

### BACKGROUND

Electrophysiology catheters are used in a variety of diagnostic and/or therapeutic medical procedures to diagnose and/or correct conditions such as cardiac arrhythmias, including for example, atrial tachycardia, ventricular tachycardia, atrial fibrillation, and atrial flutter. Cardiac arrhythmias are a leading cause of stroke, heart disease, and sudden death. The physiological mechanism of arrhythmia involves an abnormality in the electrical conduction of the heart. There are a number of treatment options for patients with arrhythmia that include medication, implantable devices, and catheter ablation of cardiac tissue.

### SUMMARY

The present disclosure generally relates to electrode assemblies for use in cardiac procedures and more particularly, to an electrode assembly that may be utilized in a cardiac mapping procedure.

In Example 1, a catheter comprises an elongate catheter body extending longitudinally from a proximal end to a distal end, an expandable electrode assembly disposed at the distal end of the catheter body, the electrode assembly comprising a housing encasing a position sensor and a plurality of flexible splines extending from the distal end of the catheter body to a distal end of the housing, and a distal cap positioned over a distal end of the housing, the distal cap being configured to receive the distal end of the housing and to further receive distal ends of flexible splines of the plurality of flexible splines in longitudinal or about longitudinal directions relative to the catheter body. Distal portions of the flexible splines provide reduced stiffness at the distal portions of the flexible splines as compared to more proximal portions of the flexible splines. The expandable electrode assembly is configured to be transitioned between a collapsed configuration suitable for delivery and an expanded configuration.

In Example 2, the catheter of Example 1, wherein the housing moves closer to the distal end of the catheter body during a transition between the collapsed configuration and the expanded configuration to allow the flexible splines to bend outwardly.

In Example 3, the catheter of Example 1, wherein, in the expanded configuration, the electrode assembly includes an about planar distally-facing sensing array surrounding the distal cap.

In Example 4, the catheter of Example 3, wherein the planar distally-facing sensing array includes at least one electrode from each of the flexible splines.

In Example 5, the catheter of Example 1, wherein the flexible splines each include at least one distally facing electrode in the expanded configuration.

In Example 6, the catheter of Example 1, wherein the distal portions of the flexible splines include narrowed necks that contribute to the reduced stiffness of the distal portions of the flexible splines.

In Example 7, the catheter of Example 6, wherein the narrowed necks include a treated surface to mitigate a risk of failure of the narrowed necks during expansion and/or contraction of the expandable electrode assembly.

In Example 8, the catheter of Example 7, wherein the treated surface includes at least one of: an electropolished surface, and a shot-peened surface.

In Example 9, the catheter of Example 7, wherein the treated surface is limited to areas including and proximate to the narrowed necks.

In Example 10, the catheter of Example 1, wherein the distal cap includes a proximally-facing recess configured to receive the distal end of the housing and the distal ends of the flexible splines.

In Example 11, the catheter of Example 10, wherein the distal ends of the flexible splines are pinched between the housing and the distal cap within the proximally-facing recess such that the distal ends of the flexible splines are effectively fixed to the housing.

In Example 12, the catheter of Example 1, wherein the flexible splines are formed from a shape memory alloy.

In Example 13, the catheter of Example 1, wherein proximal ends of the flexible splines are secured to a distal end of the catheter body.

In Example 14, the catheter of Example 1, wherein proximal ends of the flexible splines are secured to one another via a proximal band.

In Example 15, the catheter of Example 1, wherein the distal cap comprises a rounded tip having an aperture defined therein.

In Example 16, the catheter of Example 15, further comprising an adhesive disposed within the distal cap.

In Example 17, the catheter of Example 1, wherein the distal cap comprises a rounded distal end and defines an atraumatic distal tip of the catheter.

In Example 18, the catheter of Example 1, further comprising an actuation member extending from the proximal end to the distal end of the catheter body, wherein the actuation member is coupled to the expandable electrode assembly.

In Example 19, the catheter of Example 1, wherein the position sensor provides six degrees of position sensing.

In Example 20, the catheter of Example 1, wherein the distal cap serves as a distal tip electrode.

In Example 21, a catheter comprises an elongate catheter body extending longitudinally from a proximal end to a distal end, an expandable electrode assembly disposed at the distal end of the catheter body, the electrode assembly comprising a housing encasing a position sensor and a plurality of flexible splines extending from the distal end of the catheter body to a distal end of the housing, and a distal cap positioned over a distal end of the housing, the distal cap being configured to receive the distal end of the housing and to further receive distal ends of flexible splines of the plurality of flexible splines in longitudinal or about longitudinal directions relative to the catheter body, wherein distal portions of the flexible splines provide reduced stiffness at the distal portions of the flexible splines as compared to more proximal portions of the flexible splines, and wherein the expandable electrode assembly is configured to be transitioned between a collapsed configuration suitable for delivery and an expanded configuration.

In Example 22, the catheter of Example 21, wherein the housing moves closer to the distal end of the catheter body during a transition between the collapsed configuration and the expanded configuration to allow the flexible splines to bend outwardly.

In Example 23, the catheter of Example 21 or Example 22, wherein, in the expanded configuration, the electrode assembly includes an about planar distally-facing sensing array surrounding the distal cap.

In Example 24, the catheter of Example 23, wherein the planar distally-facing sensing array includes at least one electrode from each of the flexible splines.

In Example 25, the catheter of any of Examples 21 to 24, wherein the flexible splines each include at least one distally facing electrode in the expanded configuration.

In Example 26, The catheter of any of Examples 21 to 25, wherein the distal portions of the flexible splines include narrowed necks that contribute to the reduced stiffness of the distal portions of the flexible splines.

In Example 27, the catheter of Example 26, wherein the narrowed necks include a treated surface to mitigate a risk of failure of the narrowed necks during expansion and/or contraction of the expandable electrode assembly.

In Example 28, the catheter of Example 27, wherein the treated surface includes at least one of: an electropolished surface, and a shot-peened surface.

In Example 29, the catheter of any of Examples 21 to 28, wherein the distal cap includes a proximally-facing recess configured to receive the distal end of the housing and the distal ends of the flexible splines.

In Example 30, the catheter of Example 29, wherein the distal ends of the flexible splines are pinched between the housing and the distal cap within the proximally-facing recess such that the distal ends of the flexible splines are effectively fixed to the housing.

In Example 31, the catheter of any of Examples 21 to 30, wherein the flexible splines are formed from a shape memory alloy.

In Example 32, the catheter of any of Examples 21 to 31, wherein proximal ends of the flexible splines are secured to a distal end of the catheter body.

In Example 33, the catheter of any of Examples 21 to 32, wherein proximal ends of the flexible splines are secured to one another via a proximal band.

In Example 34, the catheter of any of Examples 21 to 33, further comprising an actuation member extending from the proximal end to the distal end of the catheter body, wherein the actuation member is coupled to the expandable electrode assembly.

In Example 35, the catheter of any of Examples 21 to 34, wherein the distal cap serves as a distal tip electrode.

While multiple examples are disclosed, still other examples of the present this disclosure will become apparent to those skilled in the art from the following detailed description, which shows and describes illustrative examples of the this disclosure. Accordingly, the drawings and detailed description are to be regarded as illustrative in nature and not restrictive.

### BRIEF DESCRIPTION OF THE DRAWINGS

FIG. 1 is a schematic diagram showing a catheter in the context of a system.

FIGS. 2A and 2B are schematic views of an exemplary catheter.

FIGS. 3A and 3B are isometric views of an expandable electrode assembly shown in an expanded configuration.

FIG. 4 is an isometric view of a subassembly including the cap and housing of the expandable electrode assembly of FIGS. 3A-3B.

While the this disclosure is amenable to various modifications and alternative forms, specific examples have been shown by way of example in the drawings and are described in detail below. The intention, however, is not to limit the this disclosure to the particular examples described. On the contrary, the this disclosure is intended to cover all modifications, equivalents, and alternatives falling within the scope of the this disclosure as defined by the appended claims.

### DETAILED DESCRIPTION

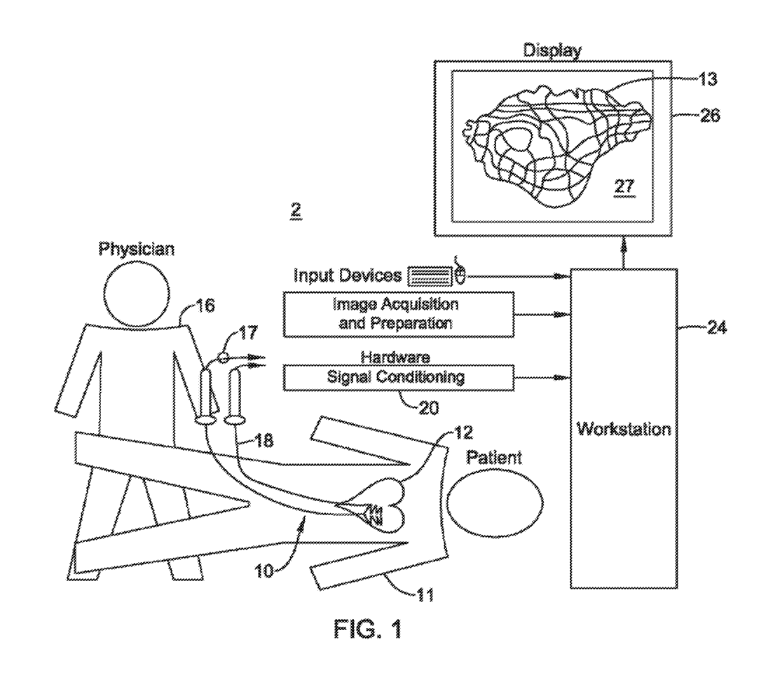

FIG. 1 is a high level, schematic view of an overall system 2 that includes a physician, a patient, catheters, including a mapping catheter 10, and related electrophysiology equipment located within an operating room. A physician 16 introduces the catheter 10 into the vasculature of the patient 11 at the patient's leg and advances it along a blood vessel ultimately, entering the patient's heart 12. Other catheters that may be used in the procedure are represented by companion catheter 18. Each catheter 10, 18 is coupled to signal conditioning hardware 20 with appropriate catheter cabling typified by catheter cable 17. The signal conditioning hardware 20 performs various interface functions applicable to the mapping, tracking, and registration procedures that are performed in conjunction with the workstation 24. If the companion catheter 18 is an ablation catheter, then conditioning hardware also forms an interface to an RF ablation unit (not illustrated).

In use, the physician looks at a computer display 26. Present on the display 26 is a substantial amount of information. A large window presents an image of the heart chamber 13 along with an image of the catheter 10. The physician will manipulate and control the catheter 10 based in part on the images and other data presented on the display 26. The image 27 seen in FIG. 1 is schematic and depicts the distal array of the deployed catheter 10 occupying a small portion of the heart chamber 13 volume. The representation of the heart chamber 13 may use color, wire frame, or other techniques to depict the structure of the heart chamber 13 and to simultaneously portray electrical activity of the patient's heart. In some cases, it may be useful to display chamber geometry, catheter location, and electrical activity in an integrated fashion on the display 26. In use, the physician will observe the display 26 and interact with the workstation processing unit 24 and the catheters 10 and 18, to direct a medical procedure such as, for example, a cardiac mapping procedure.

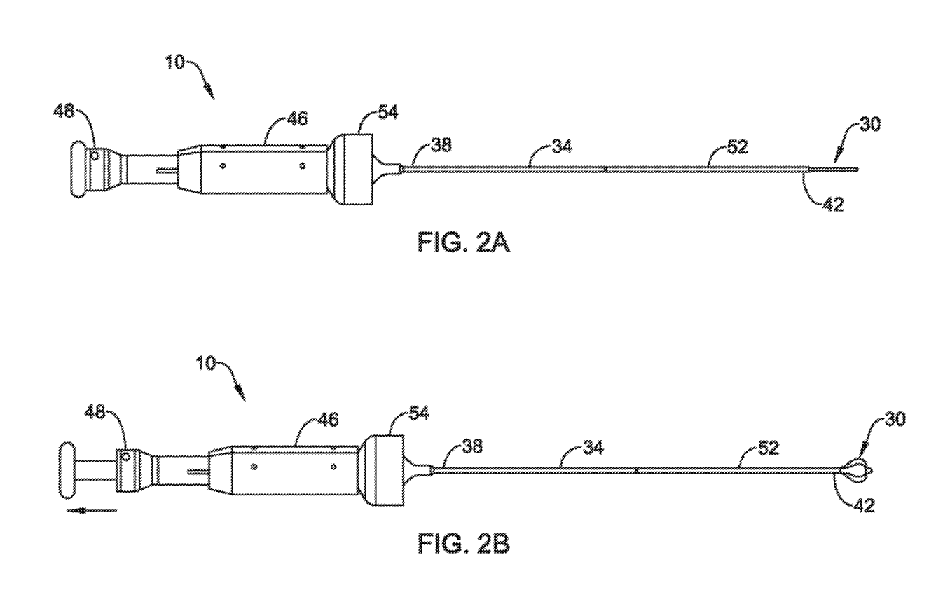

FIGS. 2A and 2B are schematic views of an exemplary intravascular catheter 10. In some cases, the catheter 10 may be used to map electro-anatomical characteristics of the heart in a cardiac mapping procedure. The mapping procedure may be an in-contact mapping or a non-contact mapping procedure. The catheter 10 may be deployed at a target location within a patient's heart, placing multiple electrodes in a known spatial configuration. Electrode stability and the known spatial geometry of the electrodes may improve the accuracy of the mapping device. Alternatively, the catheter 10 may be used in an ablation procedure. These are just some examples.

As shown in FIGS. 2A and 2B, the catheter 10 includes an elongate catheter body 34 extending from a proximal end 38 to a distal end 42. In addition, the catheter body 34 may include a lumen (not shown) extending there through, but this is not required in all examples. The catheter body 34 may have sufficient flexibility so as to navigate the tortuous pathways of a patient's vasculature system. The catheter 10 can include a handle assembly 46 coupled to the proximal end 38 of the catheter body 34. A physician may manipulate the handle assembly 46 to deliver, steer, rotate, deploy and/or deflect the catheter 10 when performing a medical procedure.

Additionally, as shown in FIGS. 2A and 2B, the catheter 10 may include an expandable electrode assembly 30 including one or more electrodes that may be used for cardiac mapping or diagnosis, ablation and/or other therapies involving the application of electrical energy to a patient's heart. In some cases, the handle assembly 46 may include a first actuation mechanism 48 that may be manipulated to transition the expandable electrode assembly 30 from a collapsed configuration (shown in FIG. 2A) suitable for delivery of the catheter 10 to a target location within a patient's body (e.g. the heart) and an expanded configuration (shown in FIG. 2B) suitable for use in a diagnostic procedure and/or delivery of a therapy. In some cases, the actuation mechanism 48 may include a pull wire that may be coupled to the expandable electrode assembly 30 that, when actuated in a proximal direction as indicated by the arrow shown in FIG. 2B, causes the expandable electrode assembly 30 to transition from the collapsed configuration to the expanded configuration. In other cases, the actuation mechanism 48 may include a retractable sheath that, when retracted in a proximal direction as indicated by the arrow shown in FIG. 2B, may permit the expandable electrode assembly 30 to self-expand from the collapsed configuration to the expanded configuration. These are just some examples of exemplary actuation mechanisms that may be utilized to facilitate expansion of the expandable electrode assembly 30 when the catheter 10 is in use. In some cases, the catheter body 34 may include a deflectable distal portion 52 that a physician may manipulate using a second actuation mechanism 54 provided in the handle assembly 46 to position the electrode assembly 30 nearer or adjacent to tissue of interest.

As shown in FIGS. 2A and 2B, the expandable electrode assembly 30 is capable of being transitioned form a generally cylindrical, collapsed configuration suitable for delivery of the catheter 10 and the electrode assembly 30 to a target location within the patient's heart and an expanded configuration suitable for use in a desired cardiac procedure such as, for example, a mapping or ablation procedure.

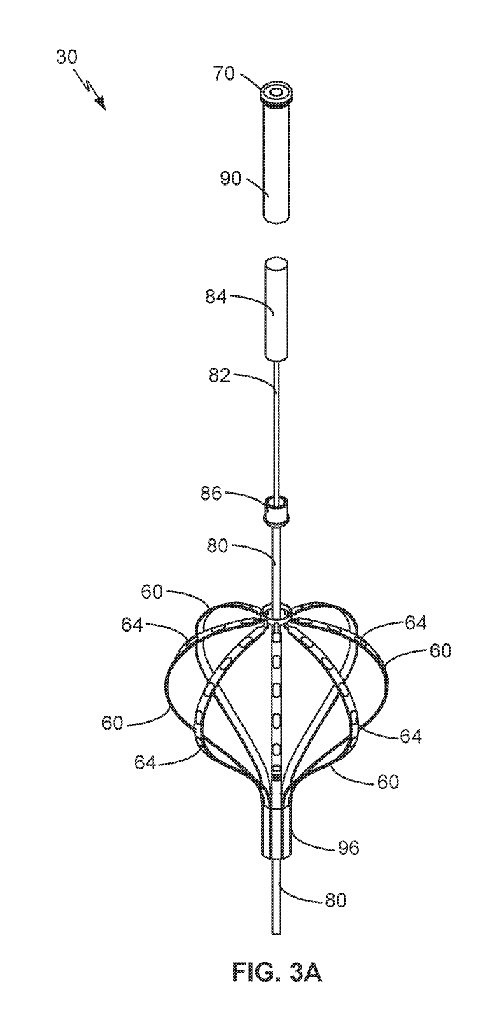

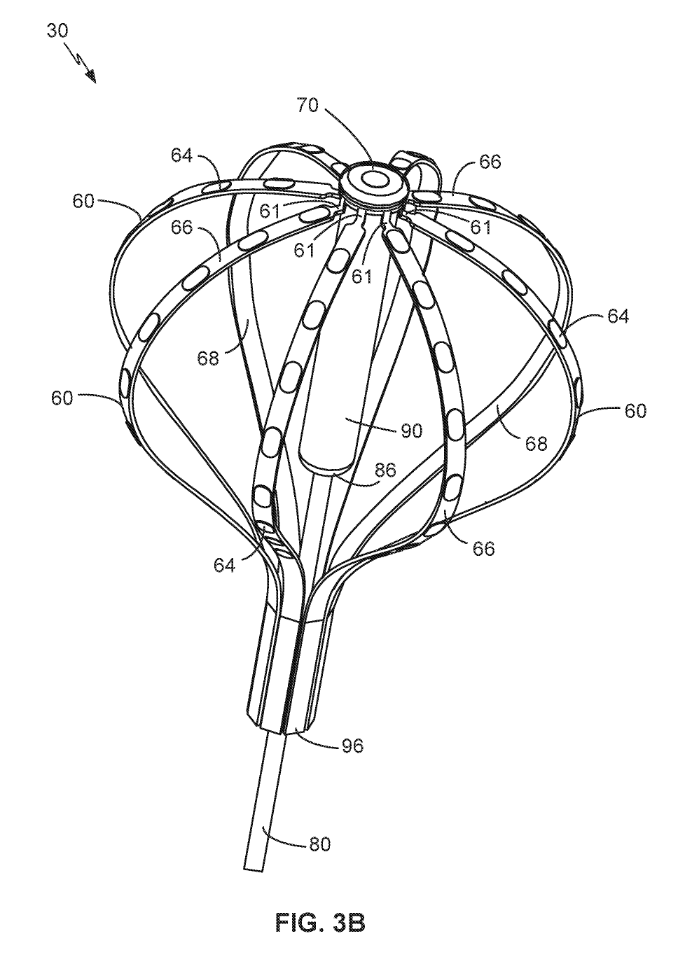

FIGS. 3A and 3B show different views of an exemplary expandable electrode assembly 30 mounted on the distal end of deployment shaft 80. The electrode assembly 30 includes flexible splines 60 mounted to a housing 90 encasing a position sensor 84, such as a position sensor with six degrees of position sensing. Sensor cap 86 covers a proximal opening of the housing 90 to enclose the position sensor 84 within the housing.

The distal ends of the flexible splines 60 are mounted to housing 90 with distal cap 70, whereas the proximal ends of flexible splines 60 are secured to proximal band 96 about deployment shaft 80. For example, the proximal ends of flexible splines 60 may be secured to a distal end of the catheter body 34 (not shown in FIGS. 3A-3B). Distal ends of the flexible splines 60 form narrowed necks 61.

Distal portions of the flexible splines 60 provide reduced stiffness at the distal portions of the flexible splines 60 as compared to more proximal portions of the flexible splines 60. The reduced stiffness at the distal ends of the flexible splines 60 are provided all or in part by the narrowed necks 61. In some or all of the flexible splines 60, the narrowed necks 61 each have a smaller cross sectional area as compared to more proximal portions of the flexible splines 60. These smaller cross sectional areas present a lower moment of resistance than the larger cross sectional areas of more proximal portions of the flexible splines 60. In some examples, the narrowed necks 61 may have about the same thicknesses as more proximal portions of the flexible splines 60, whereas narrowed necks 61 may have smaller widths than the more proximal portions of the flexible splines 60.

The reduced stiffness at the distal ends of the flexible splines 60 allows the flexible splines 60 to bend asymmetrically to provide the about planar distally-facing sensing array surrounding distal cap 70. This configuration of flexible splines 60 may provide improved sensing capabilities in the distal direction for catheter 10 as compared to other designs in which a distal end of the catheter assembly extends beyond sensing electrodes and/or other designs in which distally located electrodes 64 do not face distally.

The flexible splines 60 may be formed from a metal material with consistent material properties throughout flexible splines 60 such that the moment of resistance of flexible splines 60 only varies substantially based on the cross-sectional areas about the lengths of the flexible splines 60. In some examples, the flexible splines 60 may be formed from a shape memory alloy, such as nitinol.

The metal material of the flexible splines 60 may include a treated surface to mitigate a risk of failure of the narrowed necks during expansion and/or contraction of the expandable electrode assembly. For example, the treated surface may include an electropolished surface, a shot-peened surface and/or another surface treatment. In some examples, the treated surface may be limited to areas including and proximate to the narrowed necks 61. In other examples, the treated surface may include substantially all exterior surfaces of a metallic element of the flexible splines 60.

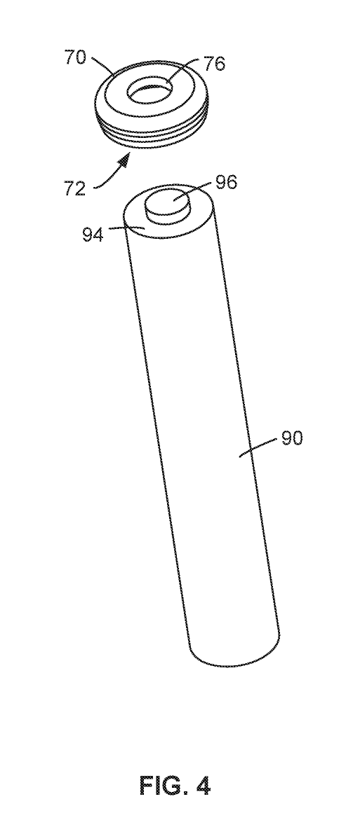

FIG. 4 illustrates a subassembly of electrode assembly 30 including a cap 70 and the housing 90 of the expandable electrode assembly 30. Distal cap 70 includes a proximally-facing recess 72 to receive a distal end of housing 90. Proximally-facing recess 72 also receives the distal ends of the flexible splines 60 in a longitudinal or about longitudinal directions relative to the catheter body 34. For example, as best shown in FIG. 3B, the distal ends of the flexible splines 60 may be pinched between the housing 90 and the distal cap 70 within the proximally-facing recess 72 such that the distal ends of the flexible splines 60 are effectively fixed to the housing 90. In some examples, the distal ends of the flexible splines 60 may be fixed to the housing 90 without welding and/or without any adhesive between the distal ends of the flexible splines 60 and the housing 90 or the distal cap 70. Alternatively, an adhesive may be utilized to the distal ends of the flexible splines 60 to secure the distal ends of the flexible splines 60 to housing 90 within the proximally-facing recess 72. In the particular example shown, the housing 90 includes notched recessed surface 94 with a protrusion 96 configured to mate with distal aperture 76 of distal cap 70.

Distal cap 70 may be rounded such that it provides the catheter 10 with an atraumatic distal tip. In addition, distal cap 70 may include a distal aperture 76, but this is not required. The distal aperture 76 may facilitate an introduction of an adhesive or other suitable potting material that may be provided as a secondary means of securing the distal ends of the flexible splines 60 within recess 72. In some cases, during construction of any one of the electrode assemblies, a cylindrical tube, plug, or gasket may be inserted into the interior cavity of the distal cap to seal any remaining gaps between the distal ends of the flexible splines 60, housing 90 and distal cap 70 within proximally-facing recess 72 subsequent to assembly. Alternatively, the distal end of the distal cap 70 may be solid.

Each of the flexible splines 60 are secured to proximal band 96 at their proximal ends. For example, the flexible splines 60 may be formed integrally with one another as a unitary component with the flexible splines 60 connected at proximal band 96. In some of such examples, splines 60 may be manufactured using techniques disclosed in U.S. Pat. App. Pub. No. 2015/0342491 by Marecki et al., the entire contents of which is incorporated by reference herein. The proximal band 96 may provide electrical connection to each of the flexible splines 160. For example, the flexible splines 60 may each include the flexible printed circuits with traces connected to the electrodes 64. The traces may terminate to pads bonded on an inner and/or outer surface of proximal band 96. In addition, the proximal band 96 may be utilized to couple to the electrode assembly 30 to the distal end 32 of the catheter body 34.

Flexible splines 60 which may be capable of being flexed outwardly and away from a longitudinal axis of the electrode assembly 30 via a control cable 82 (FIG. 3A) operated via actuation mechanism 48. In this manner, in some cases, as discussed herein, an actuation mechanism may be utilized to transition the electrode assembly 30 including the two or more flexible splines 60 from the collapsed configuration (FIG. 2A) to the expanded configuration (FIG. 2B). In other cases, the flexible splines 60 may be incorporate a shape memory material that may facilitate self-expansion of the flexible splines 60 and consequently, the electrode assembly 30, from the collapsed configuration to the expanded configuration. The flexible splines 60 may be relatively stiff such that the electrode assembly 30 may be expanded into a known, reproducible shape capable of retaining a known spatial geometry when in use which, in some cases, may be aided by the incorporation of a shape-memory material or other stiff polymeric material such as, for example, a nickel-titanium alloy, or a polyimide or PEEK into the flexible splines 60. Alternatively, depending upon the desired application, the flexible splines 60 may be fabricated such that they are somewhat compliant so as to conform to a surface of a patient's heart when placed into intimate contact with the surface of the patient's heart.

The expandable electrode assembly 30 may include a number of electrodes 64 located on each of the flexible splines 60 forming an electrode array. In many cases, the electrodes 64 may be sensing electrodes. In addition, the electrode assembly 30 may include at least some current injecting locator electrodes. The locator electrodes may be positioned diametrically opposed to each other on the meridian of the expanded electrode assembly 30. The electrode assembly 30 may also include a tip electrode which may be used for cardiac stimulation, ablation or as a locator electrode.

In particular, the electrodes 64 on distal portions of flexible splines 60 may form an about planar distally-facing (forward-facing) sensing array surrounding distal cap 70. To transition from a collapsed configuration in which flexible splines 60 are generally flat and the expanded configuration, housing 90 and the distal ends of the flexible splines 60 may move closer to the proximal band 96, which may be fixedly mounted on the distal end of the catheter body 34, such that flexible splines 60 bend outwardly. The reduced stiffness at the distal ends of the flexible splines 60 provided by narrowed necks 61 allows the flexible splines 60 to bend asymmetrically to provide the about planar distally-facing sensing array surrounding distal cap 70. This configuration of flexible splines 60 may provide improved sensing capabilities in the distal direction for catheter 10 as compared to other designs in which a distal end of the catheter assembly extends beyond sensing electrodes and/or other designs in which distally located electrodes 64 do not face distally.

Each electrode 64 may be electrically connected to the cabling in the handle assembly 46 via proximal band 96. In some cases, the signal from each individual electrode may be independently available at the hardware interface 20. This may be achieved by passing a conductor for each electrode through a connection cable extending within the catheter body 34. As an alternative, the signals may be multiplexed to minimize the number of conductors.

The electrodes 64 may have a uniform and symmetrical distribution throughout the expandable electrode assembly 30. In other cases, the electrodes 64 may have an asymmetrical distribution throughout the expandable electrode assembly 30. Certain electrode distributions may be advantageous for non-contact cardiac mapping, while others may be more suited for contact mapping. The number of electrodes 64 distributed throughout the electrode assembly 30 and the stability of the shape of electrode assembly 30, when expanded, may affect the overall performance of the mapping system.

The electrodes 64 may be located on the outer surfaces 66 of each or the splines 60, the inner surfaces 68 of each of the splines 60, or both the outer and inner surfaces 66, 68 of each of the flexible splines 60. In some cases, up to sixty-four sensing electrodes 64 may be distributed over and along the various splines 60. Depending upon the application, the electrode assembly 30 may include fewer or greater than sixty-four electrodes. In some cases, the electrodes 64 may form a number of bipolar electrode pairs. The bipolar electrode pairs may be formed between two adjacent electrodes located on the same surface (inner or outer surface) of a spline, between two electrodes located on adjacent splines, or between a first electrode located on an outer surface opposite a second electrode located on an inner surface of a spline. In some cases, all of the electrodes 64 located on the flexible splines 60 may be paired together to form a plurality of electrode pairs distributed along the length of the individual flexible splines 60. Up to thirty-two bipolar electrode pairs may be distributed throughout the electrode assembly 30 for a total of up to sixty-four electrodes 64 depending upon the overall size and geometry of the electrode assembly 30. However, it is contemplated that the electrode assembly 30 may be configured such that it is capable of carrying fewer or greater than thirty-two bipolar electrode pairs, depending upon the overall size and geometry of the electrode assembly 30 and the desired application.

Each of the flexible splines 60 may extend from a distal end 42 of the catheter body 34 to a distal cap 70. The distal cap 70 may have a rounded distal end, and may define an atraumatic distal tip of the catheter 10. In some cases, the distal cap 70 may serve as a distal tip electrode, but this is not required in all examples.

Various modifications and additions can be made to the examples discussed without departing from the scope of this disclosure. For example, while the examples described above refer to particular features, the scope of this disclosure also includes examples having different combinations of features and examples that do not include all of the described features. Accordingly, the scope of this disclosure is intended to embrace all such alternatives, modifications, and variations as fall within the scope of the claims, together with all equivalents thereof.

What is claimed is:

1. A catheter comprising:
an elongate catheter body extending longitudinally from a proximal end to a distal end;
an expandable electrode assembly disposed at the distal end of the catheter body, the electrode assembly comprising a housing encasing a position sensor and a plurality of flexible splines extending from the distal end of the catheter body to a distal end of the housing; and
a distal cap positioned over a distal end of the housing, the distal cap being configured to receive the distal end of the housing and to further receive distal ends of flexible splines of the plurality of flexible splines in longitudinal or about longitudinal directions relative to the catheter body,
wherein distal portions of the flexible splines provide reduced stiffness at the distal portions of the flexible splines as compared to more proximal portions of the flexible splines, and
wherein the expandable electrode assembly is configured to be transitioned between a collapsed configuration suitable for delivery and an expanded configuration.

2. The catheter of claim 1, wherein the housing moves closer to the distal end of the catheter body during a transition between the collapsed configuration and the expanded configuration to allow the flexible splines to bend outwardly.

3. The catheter of claim 1, wherein, in the expanded configuration, the electrode assembly includes an about planar distally-facing sensing array surrounding the distal cap.

4. The catheter of claim 3, wherein the planar distally-facing sensing array includes at least one electrode from each of the flexible splines.

5. The catheter of claim 1, wherein the flexible splines each include at least one distally facing electrode in the expanded configuration.

6. The catheter of claim 1, wherein the distal portions of the flexible splines include narrowed necks that contribute to the reduced stiffness of the distal portions of the flexible splines.

7. The catheter of claim 6, wherein the narrowed necks include a treated surface to mitigate a risk of failure of the narrowed necks during expansion and/or contraction of the expandable electrode assembly.

8. The catheter of claim 7, wherein the treated surface includes at least one of:
an electropolished surface; and
a shot-peened surface.

9. The catheter of claim 7, wherein the treated surface is limited to areas including and proximate to the narrowed necks.

10. The catheter of claim 1, wherein the distal cap includes a proximally-facing recess configured to receive the distal end of the housing and the distal ends of the flexible splines.

11. The catheter of claim 10, wherein the distal ends of the flexible splines are pinched between the housing and the distal cap within the proximally-facing recess such that the distal ends of the flexible splines are effectively fixed to the housing.

12. The catheter of claim 1, wherein the flexible splines are formed from a shape memory alloy.

13. The catheter of claim 1, wherein proximal ends of the flexible splines are secured to a distal end of the catheter body.

14. The catheter of claim 1, wherein proximal ends of the flexible splines are secured to one another via a proximal band.

15. The catheter of claim 1, wherein the distal cap comprises a rounded tip having an aperture defined therein.

16. The catheter of claim 15, further comprising an adhesive disposed within the distal cap.

17. The catheter of claim 1, wherein the distal cap comprises a rounded distal end and defines an atraumatic distal tip of the catheter.

18. The catheter of claim 1, further comprising an actuation member extending from the proximal end to the distal end of the catheter body, wherein the actuation member is coupled to the expandable electrode assembly.

19. The catheter of claim 1, wherein the position sensor provides six degrees of position sensing.

20. The catheter of claim 1, wherein the distal cap serves as a distal tip electrode.
